(19) United States
(12) Patent Application Publication
(10) Pub. No.: US 2024/0145098 A1
(43) Pub. Date: May 2, 2024

(54) PREDICTIVE MODELING FRAMEWORK FOR RISK ASSESSMENT

(71) Applicant: Falcon Inc., Chicago, IL (US)

(72) Inventor: Elizabeth Falcon, Dunlap, IL (US)

(73) Assignee: Falcon Inc., Chicago, IL (US)

(21) Appl. No.: 18/494,241

(22) Filed: Oct. 25, 2023

Related U.S. Application Data
(60) Provisional application No. 63/381,155, filed on Oct. 27, 2022.

(51) Int. Cl. G16H 50/30; G16H 20/70

(52) U.S. Cl. CPC G16H 50/30; G16H 20/70

(57) ABSTRACT

A predictive modeling framework for various correctional and community areas. A central repository stores raw data representative of a plurality of attributes associated with an inmate's risk of suicide. A data summary maps the raw data, which is extracted from a plurality of source systems, to a predetermined data framework that relates the attributes and risk factor loads for the target area of interest. A machine-learned model executed on the data summary generates a predictive risk profile capturing behavioral patterns indicative of a potential predefined risk associated with the target area of interest. The risk assessment is based on the predictive risk profile generated by the machined-learned model.

### CROSS-REFERENCE TO RELATED APPLICATION

The present application claims the benefit of U.S. Provisional Application No. 63/381,155, filed Oct. 27, 2022, the entire disclosure of which is incorporated herein by reference.

### BACKGROUND

Deaths by suicide among inmates have long been shown to occur at much higher rates than among general populations of similar ages and genders. Known approaches to reduce suicide risk in jails and prisons include risk assessment and management for individual inmates, and targeting modifiable risk factors. Improving assessments and interventions to reduce suicide risk among incarcerated persons is needed.

### SUMMARY

Aspects of the present disclosure strengthen and elevate the principles for assessing and screening suicide potential in a jail and prison setting with the goal of increasing reliability, predictability, and effectiveness of suicide risk assessments and prevention tools.

In an aspect, a computer-implemented method generates a risk assessment in a target area of interest. The method includes extracting, from a plurality of source systems, raw data representative of a plurality of attributes associated with the target area of interest and mapping the raw data extracted from the source systems to a data summary. The data summary is a predetermined data framework relating the attributes and risk factor loads for the target area of interest. The method also includes executing a machine-learned model on the data summary to generate a predictive risk profile capturing behavioral patterns indicative of a potential predefined risk associated with the target area of interest and generating the risk assessment in the target area of interest based on the predictive risk profile generated by the machined-learned model.

In another aspect, a predictive modeling system for generating a risk assessment in a target area of interest includes a central repository storing raw data representative of a plurality of attributes associated with the target area of interest. A data summary maps the raw data, which is extracted from a plurality of source systems, to a predetermined data framework that relates the attributes and risk factor loads for the target area of interest. The system also includes a machine-learned model that, when executed on the data summary, generates a predictive risk profile capturing behavioral patterns indicative of a potential predefined risk associated with the target area of interest. The risk assessment in the target area of interest is based on the predictive risk profile generated by the machined-learned model.

Other objects and features will be in part apparent and in part pointed out hereinafter.

### BRIEF DESCRIPTION OF THE DRAWINGS

FIG. 1 is a block diagram illustrating an example process for predictive modeling according to an embodiment.

FIG. 2 further illustrates the example process of FIG. 1.

FIG. 3 is an entity-relationship diagram illustrating an example representation of data modeling for use in the process of FIGS. 1 and 2.

Corresponding reference characters indicate corresponding parts throughout the drawings.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

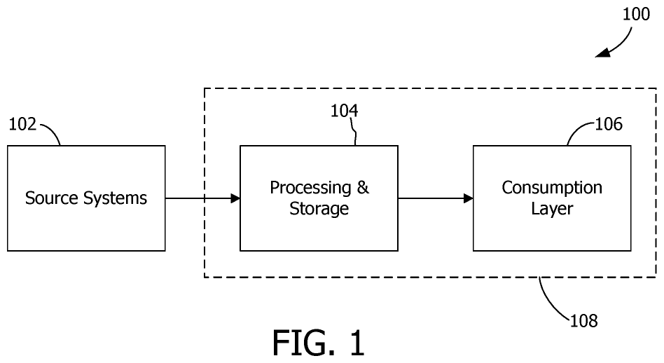

FIG. 1 is a block diagram illustrating an example predictive modeling process 100 embodying aspects of the present disclosure. In an embodiment, the process 100 provides, for example, suicide risk assessment and screening. The process 100 capitalizes on a predictive modeling framework that strengthens and elevates the principles for assessing and screening suicide potential in a jail or prison setting with the goal of increasing reliability, predictability, and effectiveness of suicide risk assessments and prevention tools. To achieve this goal, process 100 utilizes a machine-learned model, which leverages Artificial Intelligence (AI) technology as described below through the hyper-convergence of AI, Deep Learning (DL), and Machine Learning (ML) merged with data (of high-risk populations) gathered from jails across identified County and State Jurisdictions throughout the U.S.

In FIG. 1, source systems 102 hold the data that maps on to a data summary (described below). The source systems 102 are data sources relating to, for example, a jail facility. Processing and storage 104 represents a reusable pipeline configured to extract the data from source systems 102 and to store the extracted data as a sync in the predictive modeling framework. A consumption layer 106 provides the ML-powered framework for analyzing the data from the processing and storage 104 and a mechanism that permits downstream applications to consume the data. In an embodiment, a computer 108 executes processor-executable instructions for performing the functions of processing and storage 104 and consumption layer 106.

The ML-powered framework of the consumption layer 106 is configured to capture behavioral patterns of suicidal behavior based on established and well-researched risk factors for suicide attempters and completers in jails and prisons. Machine Learning frameworks represent an advanced analytics domain which is mathematically and statistically intensive in computing probability patterns. The ML model consumption layer 106 is applied to suicide assessment in jails and prisons and enables process 100 to:
a) Ingest structured historical data from jail/prison facilities pertinent to an inmate's medical and mental health histories;
b) Assess suicide case profiles based on chronic, acute, and protective risk factors;
c) Assess and prioritize intervention efforts across jails and prisons based on associated risk or severity of risk; and
d) Monitor changes in risk profiles, across facilities in identified County and State Jurisdictions across the U.S.

The hyperconvergence of data via the ML framework of consumption layer 106 provides data-powered dashboards configured to be displayed on the computer 108 or deployed to jail and prison facilities across the nation via engineered software executing, for example, a web service. The software consumes risk data from new cases and reacts by providing a predictive risk profile that can assist custody and healthcare staff to more effectively and proactively identify and intervene with potential high-risk inmates and suicide attempters.

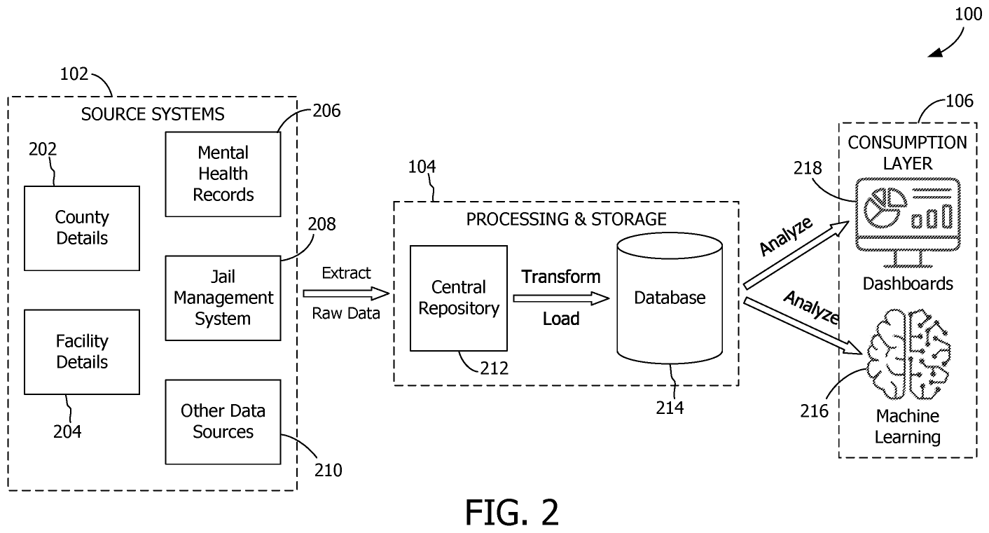

FIG. 2 further illustrates the example process 100 embodying aspects of the present disclosure. The process 100 begins by creating a data framework, or summary. In an embodiment, the data summary comprises a structured, hierarchical data framework with interwoven communication and relationships between risk factor categories, attributes, and risk factor loads. For any given model (e.g., Suicide Risk Assessment, Violence Risk Assessment, etc.), process 100 promulgates a data summary on factor loads and attributes for each target area of interest. In an embodiment for suicide risk prevention screening, for example, the data is promulgated on measurements of acute, chronic, and protective risk factors and attributes for correctional residents or inmates to identify those at risk for suicide attempts or completions within a jail setting. Using an Entity Relationship Diagram (ERD) model, described below, the data summary for this predictive model provides a mapping out of the exact risk factor loads, attributes, data categories, relationships, and hierarchies of an individual at risk for suicide in a jail setting.

With the data summary completed, process 100 collects data at from cases within the “lived” environment. For example, for suicide risk prevention screening of inmates, the exact sample group are inmates within a jail system. Thus, a sample of data representative of the appropriate demographics is collected from jail systems from across the nation. Data source systems within each jail, such as sample size, target populations, jail size, county size, and demographics, are carefully considered and predetermined prior to initiating data collection. As shown in FIG. 2, example data sources of source systems 102 include county details 202, facility details 204, mental health records 206, jail management system data 208, and other sources of data 210.

The processing and storage 104 performs data extraction of raw data from source systems 102 to a central repository 212. The exact data to be mapped on to the predetermined data framework, or summary, is extracted from the jail source systems 102 through a reusable pipeline that can be stored as a sync to the data storage environment (i.e., data is synchronized and flows directly to the central repository 212). According to an embodiment, processing and storage 104 receives the data at central repository 212 and then processes and converts the received data into the predefined data structure (data summary) stored in a database 214.

Referring further to FIG. 2, consumption layer 106 processes the data summary for downstream consumption. Machine learning 216 in the illustrated embodiment applies advanced analytics and algorithms to the data summary through AI and ML technology. For example, ML 216 is applied to weigh in on each parameter or attribute for capturing life or behavior patterns or trends and identifying potential risks to red flag an inmate as an attempter or completer of suicide. Once ML 216 analyzes the data summary to identify and analyze the potential risk, a purpose-built dashboard 218 highlights the assessment to a user. In an embodiment, the dashboard 218 provides drill down capabilities and real time visibility to the jails to identify potential risk inmates.

The predictive model embodying aspects of the present disclosure is “self-training” to confer with every new incremental data loaded on from the facility level. For every new data point received at the facility level, it is automatically synced, pushed through, analyzed, and the model is immediately able to identify potential risk inmates. The dashboard 218 can be configured to be fed available data from correctional facilities across the U.S., allowing ML 216 to learn and visualize trends and segments across facilities over time.

The dashboard 218 is configured to be deployed throughout jails across the U.S. and incorporated with screening tools built into each facility's Jail Management System (JMS). In use, staff will administer this tool and input inmate responses into the JMS for every inmate that is admitted into the facility. The source system 102 receives this information through a pipeline and pushes the information through predictive modeling process 100 as described above. In an embodiment, processing time for newly received information to be pushed through and for process 100 to deliver the output is estimated to be about 5 minutes.

As described above, the predictive modeling framework in accordance with embodiments of the present disclosure begins with a data framework or data summary predetermined for use in process 100. For example, for suicide risk prevention screening, the data is promulgated on measurements of acute, chronic, and protective risk factors and attributes for correctional residents or inmates to identify those at risk for suicide attempts or completions within a jail setting. Using the ERD model, the data summary provides a mapping out of the exact risk factor loads, attributes, data categories, relationships, and hierarchies of an individual at risk for suicide in a jail setting. Tables I and II, below, are examples of Data Summaries in accordance with embodiments of the present disclosure.

TABLE I
Data Summary:

| Source | | | | |
|---|---|---|---|---|
| Type | Instrument/Why | | Inmate Details/Why | |
| General | ETOH | Recency of substance | Ethnicity | Different races have |
| Details | | addiction and abuse | | different dread |
| | | | | tolerance |
| Chronic | Previous | Recency of attempts | Admission | Red flags based on |
| Risk | Attempts | shows outlook to act | Records | behavior at realization |
| Factors | | | | of arrest |
| Acute Risk | Ideation | Recency of planning | Trauma | Recency of emotional/ |
| Factors | | shows conviction | Details | physical/mental trauma |
| | | | | shows red flags |
| Protective | Sustainability | Recency of rooting | Family | Support structure has |
| Factors | Details | structures shows | | ameliorative effects |
| | | positive signs in | | |
| | | inmate's life | | |

TABLE II
Data Summary:

| Source | | | | |
|---|---|---|---|---|
| Type | Case Record/Why | | County Details/Why | |
| General | Mental | Recency of psych | Supervision | Direct vs. indirect |
| Details | Health | evaluations shows | Style | management style has |
| | Psychiatric | red flags | | massive effect in |
| | Evaluations | | | attempt |
| Chronic | Withdrawal | Recency of substance | Detainee | Low visitor interaction |
| Risk | | abuse withdrawal | Interactions | red flags an attempt |
| Factors | | symptoms | | |
| Acute | Suicide | Recent listing in | Detainee | Number of cell mates |
| Risk | Watch | suicide watch shows | Housing | is proportional to |
| Factors | | red flags | | quality of life |
| Protective | Habits & | Maintenance of | Social Behavior | Low social interaction |
| Factors | Beliefs | positive routines | | red flags an attempt |
| | | shows positive signs | | |
| | | in inmate's life | | |

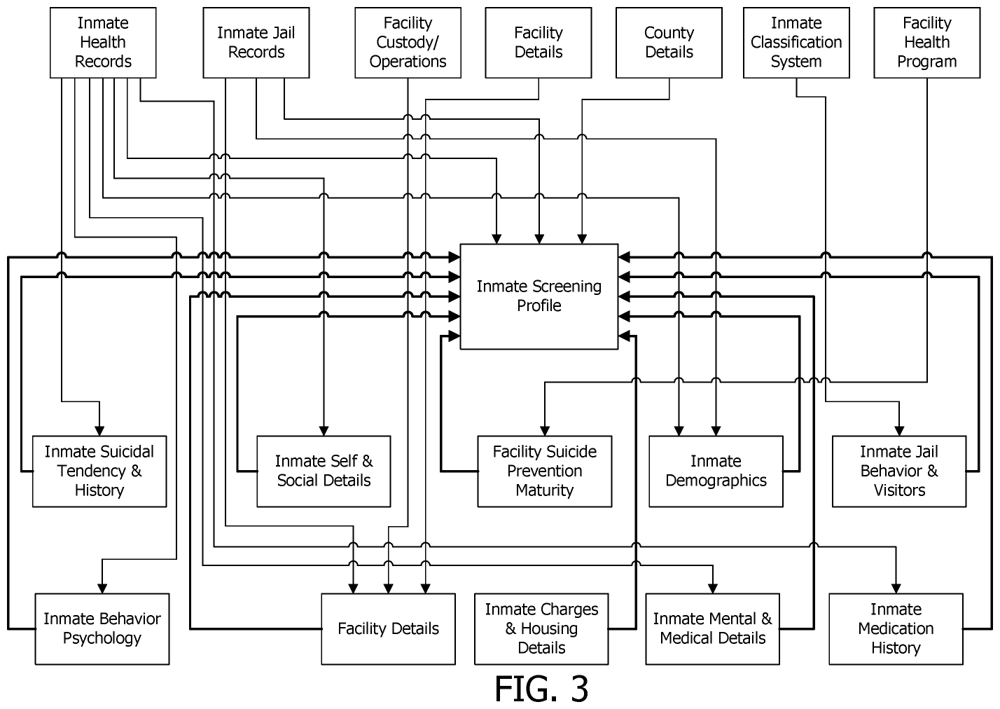

FIG. 3 is an entity-relationship diagram (ERD) illustrating an example representation of data modeling for use in the process 100 of FIGS. 1 and 2. FIG. 3 illustrates relationships and communication between data elements.

In the example of FIG. 3, data inputs include:
a) Inmate Healthcare/Mental Health Records;
b) Inmate Jail Management Systems Records;
c) Facility Custody/Operations Details (Data Source: Jail Admin);
d) Facility Details (Data Source: Jail Admin);
e) County Details (Data Source: County Admin);
f) Inmate Jail Management Systems Records/Classification System; and
g) Facility Healthcare/Mental Health Program Details.

The predictive modeling framework is described above with respect to suicide risk assessment and prevention in the context of incarcerated persons. It is to be understood that the predictive modeling framework described herein may also be applied to areas in correctional and community healthcare and mental health, public safety, and public health in accordance with embodiments of the present disclosure without deviating from the scope set forth in the claims. For example, aspects of the present disclosure are applicable to predictive modeling of various types of correctional and community violence risk assessment and prevention, including prediction-based assessment of correctional officer suicide risk and prevention, mass shootings risk and prevention, restrictive housing placement, etc.

Although described in connection with an example computing system environment, embodiments of the aspects of the disclosure are operational with other special purpose computing system environments or configurations. The computing system environment is not intended to suggest any limitation as to the scope of use or functionality of any aspect of the disclosure. Moreover, the computing system environment should not be interpreted as having any dependency or requirement relating to any one or combination of components illustrated in the example operating environment. Examples of computing systems, environments, and/or configurations that may be suitable for use with aspects of the disclosure include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

Embodiments of the aspects of the present disclosure may be described in the general context of data and/or processor-executable instructions, such as program modules, stored one or more tangible, non-transitory storage media and executed by one or more processors or other devices. Generally, program modules include, but are not limited to, routines, programs, objects, components, and data structures that perform particular tasks or implement particular abstract data types. Aspects of the present disclosure may also be practiced in distributed computing environments where tasks are performed by remote processing devices that are linked through a communications network. In a distributed computing environment, program modules may be located in both local and remote storage media including memory storage devices.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

Shared memory hardware encompasses a single memory device that stores some or all code from multiple modules. Group memory hardware encompasses a memory device that, in combination with other memory devices, stores some or all code from one or more modules.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer. For purposes of illustration, programs and other executable program components may be shown as discrete blocks. It is recognized, however, that such programs and components reside at various times in different storage components of a computing device, and are executed by a data processor(s) of the device.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTMLS (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

In operation, processors, computers and/or servers may execute the processor-executable instructions (e.g., software, firmware, and/or hardware) such as those illustrated herein to implement aspects of the disclosure. Preferably, the processor-executable instructions are stored in a memory, such as the hard disk drive, and executed by the computer. Advantageously, the computer processor has the capability to perform all operations (e.g., execute processor-executable instructions) in real-time.

The order of execution or performance of the operations in embodiments illustrated and described herein is not essential, unless otherwise specified. That is, the operations may be performed in any order, unless otherwise specified, and embodiments may include additional or fewer operations than those disclosed herein. For example, it is contemplated that executing or performing a particular operation before, contemporaneously with, or after another operation is within the scope of aspects of the disclosure.

Embodiments may be implemented with processor-executable instructions. The processor-executable instructions may be organized into one or more processor-executable components or modules on a tangible processor readable storage medium. Also, embodiments may be implemented with any number and organization of such components or modules. For example, aspects of the present disclosure are not limited to the specific processor-executable instructions or the specific components or modules illustrated in the figures and described herein. Other embodiments may include different processor-executable instructions or components having more or less functionality than illustrated and described herein.

When introducing elements of the invention or embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively, or in addition, a component may be implemented by several components.

The above description illustrates embodiments by way of example and not by way of limitation. This description enables one skilled in the art to make and use aspects of the disclosure, and describes several embodiments, adaptations, variations, alternatives and uses of the aspects of the disclosure, including what is presently believed to be the best mode of carrying out the aspects of the disclosure. Additionally, it is to be understood that the aspects of the disclosure are not limited in its application to the details of construction and the arrangement of components set forth in the following description or illustrated in the drawings. The aspects of the disclosure are capable of other embodiments and of being practiced or carried out in various ways. Also, it will be understood that the phraseology and terminology used herein is for the purpose of description and should not be regarded as limiting.

It will be apparent that modifications and variations are possible without departing from the scope of the invention defined in the appended claims. As various changes could be made in the above constructions and methods without departing from the scope of the invention, it is intended that all matter contained in the above description and shown in the accompanying drawings shall be interpreted as illustrative and not in a limiting sense.

In view of the above, it will be seen that several advantages of the aspects of the invention are achieved and other advantageous results attained.

What is claimed is:

1. A computer-implemented method of generating a risk assessment in a target area of interest, the method comprising:
extracting, from a plurality of source systems, raw data representative of a plurality of attributes associated with the target area of interest;
mapping the raw data extracted from the source systems to a data summary, the data summary comprising a predetermined data framework relating the attributes and risk factor loads for the target area of interest;
executing a machine-learned model on the data summary to generate a predictive risk profile capturing behavioral patterns indicative of a potential predefined risk associated with the target area of interest; and
generating the risk assessment in the target area of interest based on the predictive risk profile generated by the machined-learned model.

2. The method of claim 1, wherein the target area of interest comprises one or more areas within correctional and community healthcare and mental health, public safety, and public health for correctional and community violence risk assessment and prevention.

3. The method of claim 2, wherein correctional and community violence risk assessment and prevention comprises at least one of: risk assessment and prevention of suicide by an inmate; risk assessment and prevention of suicide by a correctional officer; risk assessment and prevention of a mass shooting; risk assessment and prevention of violence in a correctional environment; risk assessment and prevention of violence in a community environment; and disciplinary and behavioral violence risk assessment and prevention.

4. The method of claim 1, further comprising storing the raw data extracted from the source systems in a central repository before mapping to the data summary.

5. The method of claim 1, wherein the data summary comprises an entity-relationship diagram (ERD).

6. The method of claim 5, wherein mapping the raw data extracted from the source systems to the data summary comprises mapping the risk factor loads and the attributes to data categories, relationships, and hierarchies of an individual at risk for suicide in a jail setting as defined in the ERD.

7. The method of claim 1, further comprising generating a dashboard for visualizing a risk assessment based on the predictive risk profile.

8. The method of claim 7, further comprising deploying the dashboard to one or more facilities via a web service.

9. The method of claim 1, wherein the machine-learned model is configured to capture the behavioral patterns of suicidal behavior based on predetermined risk factors.

10. The method of claim 1, wherein mapping the raw data extracted from the source systems comprises ingesting structured historical data from one or more jail or prison facilities pertinent to medical and mental health histories of an inmate.

11. The method of claim 1, wherein the predictive risk profile comprises an assessment of suicide case profiles based on chronic, acute, and protective risk factors and an assessment of intervention efforts across jails and prisons based on associated risk or severity of risk.

12. The method of claim 1, wherein the raw data stored in the source systems comprises one or more of sample size, target populations, jail size, county size, and demographics.

13. The method of claim 1, wherein the data sources of the source systems include one or more of county details, facility details, mental health records, jail management system data, and other sources of data.

14. A predictive modeling system for generating a risk assessment in a target area of interest, the system comprising:
a central repository storing raw data representative of a plurality of attributes associated with the target area of interest, the raw data extracted from a plurality of source systems;
a data summary mapping the raw data extracted from the source systems to a predetermined data framework, the data summary relating the attributes and risk factor loads for the target area of interest; and
a machine-learned model that, when executed on the data summary, generates a predictive risk profile capturing behavioral patterns indicative of a potential predefined risk associated with the target area of interest;
wherein the risk assessment in the target area of interest is based on the predictive risk profile generated by the machined-learned model.

15. The system of claim 14, wherein the target area of interest comprises one or more areas within correctional and community healthcare and mental health, public safety, and public health for correctional and community violence risk assessment and prevention, and wherein correctional and community violence risk assessment and prevention comprises at least one of: risk assessment and prevention of suicide by an inmate; risk assessment and prevention of suicide by a correctional officer; risk assessment and prevention of a mass shooting; risk assessment and prevention of violence in a correctional environment; risk assessment and prevention of violence in a community environment; and disciplinary and behavioral violence risk assessment and prevention.

16. The system of claim 14, wherein the data summary comprises an entity-relationship diagram (ERD).

17. The system of claim 16, wherein the ERD defines a mapping of the risk factor loads and the attributes to data categories, relationships, and hierarchies of an individual at risk for suicide in a jail setting.

18. The system of claim 14, further comprising a dashboard for visualizing a risk assessment based on the predictive risk profile.

19. The system of claim 14, wherein the machine-learned model is configured to capture the behavioral patterns of suicidal behavior based on predetermined risk factors.

20. The system of claim 14, wherein the predictive risk profile comprises an assessment of suicide case profiles based on chronic, acute, and protective risk factors and an assessment of intervention efforts across jails and prisons based on associated risk or severity of risk.

21. The system of claim 14, wherein the raw data stored in the source systems comprises one or more of sample size, target populations, jail size, county size, and demographics, and wherein the data sources of the source systems include one or more of county details, facility details, mental health records, jail management system data, and other sources of data.
